(19) United States
(12) Patent Application Publication
(10) Pub. No.: US 2025/0306049 A1
(43) Pub. Date: Oct. 2, 2025

(54) APPARATUS AND METHODS FOR PROCESSING BIOLOGICAL SAMPLES

(71) Applicant: STEMCELL TECHNOLOGIES CANADA INC., Vancouver (CA)

(72) Inventor: Payam ZACHKANI, Vancouver (CA)

(21) Appl. No.: 19/234,060

(22) Filed: Jun. 10, 2025

Related U.S. Application Data
(63) Continuation of application No. PCT/CA2023/051668, filed on Dec. 15, 2023.
(60) Provisional application No. 63/387,891, filed on Dec. 16, 2022.

(51) Int. Cl. G01N 35/02; B01L 3/00; G01N 1/28; G01N 33/50

(52) U.S. Cl. CPC G01N 35/025; B01L 3/50825; G01N 1/286; G01N 33/50; G01N 2001/2873

(57) ABSTRACT

The present application relates to a cartridge for processing biological samples, such as tissue or tumour samples. The cartridge may interface with a system, such as an automated system, to process a biological sample. A cartridge and/or a system of this disclosure may be used in methods of processing a biological sample. In certain use cases, a cartridge and/or system and/or method of this disclosure may mince a biological sample. In certain use cases, a cartridge and/or system and/or method of this disclosure may dissociate a biological sample. In certain use cases, a cartridge and/or system and/or method of this disclosure may homogenize a biological sample.

### CROSS REFERENCE TO RELATED APPLICATION

This application is a continuation of Patent Cooperation Treaty (PCT) application No. PCT/CA2023/051668, having an international filing date of 15 Dec. 2023, which in turns claims the benefit of United States Provisional Patent Application No. 63/387891 filed Dec. 16, 2022, the entire content of which is hereby incorporated by reference in its entirety.

### TECHNICAL FIELD

This disclosure relates to apparatus and methods for processing biological samples, such as tissue samples. More specifically, this disclosure relates to automated apparatus and methods for processing biological samples, such as tissue samples. Still more specifically, this disclosure relates to automated apparatus and methods for dissociating and/or homogenizing biological samples, such as tissue samples.

### BACKGROUND

In various cell and molecular biology applications researchers and technicians often have a need to process biological samples prior to conducting downstream assays or analyses. In the case of cell biologists, assays are routinely conducted using cells or organelles obtained from solid tissues or tumours. In the case of molecular biologists, assays are routinely conducted using nucleic acids and proteins obtained from cells, such as those cells located within a liquid sample or a solid tissue or tumour sample.

The use of enzymes or solutions containing enzymes to obtain a suspension of cells from a tissue or tumour is widespread. Indeed, numerous commercial vendors offer reagents for this purpose. However, depending on the tissue or tumour type, enzymatic dissociation may take a prolonged period of time and/or result in incomplete dissociation and/or digest relevant materials such as proteins or glycoproteins. Less commonly, tissues and tumours may be mechanically dissociated, such as using a mortar and pestle, or a rotor and stator arrangement. However, such techniques may be limited by inconsistent cell yields, poor cell viability, and user skill.

Accordingly, there is a need for new approaches to the dissociation and/or homogenization of biological samples, such as tissues. Solutions to this problem may desirably recover high numbers of cells that are viable. In certain application it may be further desirable to dissociate a biological sample while minimizing the homogenization of cells.

### SUMMARY

The present disclosure relates to apparatus and methods for processing biological samples, such as tissue or tumour samples. More specifically, this disclosure relates to automated apparatus and methods for processing biological samples, such as tissue or tumour samples.

In one aspect of this disclosure are provided apparatus for processing a biological sample. In one embodiment, apparatus is a cartridge that may be used to process a biological sample. In one embodiment, a cartridge for processing a biological sample may comprise a cap having an upper side and an underside; a shaft rotatable relative to the cap, the shaft having a first end that cooperates with the cap and an opposed second end extending away from the underside; an agitator attached to the shaft; one or more slots in a peripheral edge of the agitator; a receptacle engageable with the underside of the cap; and one or more teeth arranged in an interior wall of the receptacle, the one or more teeth projecting toward an interior of the receptacle and respectively passing through the one or more slots as the agitator rotates about an axis defined by the shaft.

In one embodiment, the agitator is an impeller. In one embodiment, the agitator has a substantially constant radius along its length (in the direction of a longitudinal axis of the shaft). In one embodiment, the agitator flares radially wider in a direction from the first end toward the second end. In one embodiment, the agitator is integral with the shaft.

In one embodiment, a cartridge of this disclosure may further comprise a plurality of teeth. In one embodiment, the plurality of teeth are annularly arranged in at least a first bank of teeth and a second bank of teeth. In one embodiment, the first bank of teeth project further toward the interior of the receptacle than the second bank of teeth. In one embodiment, the first bank of teeth is positioned radially outward of the second bank of teeth.

In one embodiment, the one or more teeth are arranged in a bottom wall of the receptacle.

In one embodiment, a cross section of the one or more teeth taken in a plane parallel to the interior wall is an ellipse. In one embodiment, an eccentricity of the ellipse is greater than 0 and less than 1. In one embodiment, the eccentricity of the ellipse is greater than 0.5.

In one embodiment, the one or more teeth terminate in a tapered edge. In one embodiment, the tapered edge is smooth. In one embodiment, the tapered edge is serrated.

In one embodiment, a cartridge of this disclosure may further comprise an alignment feature in the bottom wall of the receptacle for engaging a tip of the second end of the shaft.

In one embodiment, the cartridge is a dissociation cartridge for dissociating the tissue sample. In one embodiment, the cartridge is a homogenization cartridge for homogenizing the tissue sample.

In another aspect of this disclosure are provide assembled cartridges comprising one or more of the features as described above, wherein the cap is engaged with the receptacle.

In another aspect of this disclosure are provided systems for processing a biological sample. In one embodiment, the systems are automated. In one embodiment, a system of this disclosure may comprise a base having one or more receiving areas for respectively receiving an assembled cartridge as described herein, at least one rotatable spindle engagable with the shaft of the assembled cartridge, one a motor for rotating the spindle, and at least one processor or microprocessor configured to output a processing protocol or instruction to at least the motor (via a controller or a micro(controller)).

In one embodiment, a system of this disclosure may further comprise venting. In one embodiment, a system of this disclosure may further comprise ducting in fluid communication with an internal cavity of the base. In one embodiment, a system of this disclosure may further comprise one or more fans to move air within system. In one embodiment, the one or more fan is configured to draw air into the duct and through the venting (out of the system).

In one embodiment, a cartridge (e.g. an assembled cartridge) may be received within a receiving area. In one embodiment, a receiving area comprises a bore circumscribed by a bore wall.

In one embodiment, a system of this disclosure may further comprise a Peltier module for establishing or modifying a temperature of the bore wall. In one embodiment, the processing protocol comprises establishing or modifying a temperature of the bore wall.

In one embodiment, the processing protocol comprises setting the speed and/or direction and/or duration of rotation of the motor (and at least one spindle and associated shaft/agitator).

In one embodiment, the processing protocol or instruction is selected or input via a graphical user interface.

In one embodiment, a system of this disclosure may further comprise a sensor downstream of the at least one processor or microprocessor, the sensor relaying a feedback signal to the at least one processor or microprocessor.

In one embodiment, the spindle is movable or biasable from a first retracted position to a second extended position.

In another aspect of this disclosure are provided methods for processing a biological sample. Methods of this disclosure may comprise providing a biological sample in a cartridge (as described herein), executing a processing protocol, and yielding a processed biological sample that is reduced in complexity.

In one embodiment, executing a processing protocol comprises rotating an agitator to direct a biological sample toward and into contact with one or more teeth configured in a cartridge. In one embodiment, rotating an agitator comprises rotation for a defined period of time and/or a defined speed of rotation. In one embodiment, rotating an agitator comprises unidirectional rotation. In one embodiment, rotating an agitator comprises reversing a direction of agitator rotation at least once during a processing protocol. In one embodiment, rotating an agitator comprises oscillating a direction of agitator rotation during a processing protocol.

In one embodiment, executing a processing protocol comprises at least one incubation. In one embodiment, executing a processing protocol comprises more than one incubation. In one embodiment, incubation(s) during a processing protocol are performed at a present temperature, ranging between 4° C. and 55° C.

In one embodiment, a biological sample reduced in complexity ranges along the continuum from minced to dissociated to homogenized.

Methods of this disclosure may further comprise post-processing the processed biological sample. By way of example, post-processing may comprise filtration, washing, myelin removal, DNase treatment, nucleic acid extraction, protein isolation, immunostaining, debris removal, cell separation/enrichment, and/or removal/lysis of red blood cells.

Methods of this disclosure may further comprise subjecting a processed or post-processed biological sample to further downstream analysis. By way of example, further downstream analysis may comprise: nucleic acid analysis by PCR, qPCR, RT-qPCR, sequencing or high throughput sequencing, or the like; protein analysis by Western, immunostaining, proteomics, or the like; or cell analysis by flow cytometry, or the like.

In one embodiment, providing a biological sample in cartridge (of this disclosure) may comprise interfacing the cartridge with a system, as described hereinabove. Thus, methods of this disclosure may be automated methods.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a perspective front view of an assembled cartridge wherein the cap is engaged with the receptacle.

FIG. 2 shows a perspective front view of a cartridge cap disengaged from a receptacle (A), and a perspective bottom view of the underside of a cartridge cap disengaged from a receptacle (B).

FIG. 3 shows a perspective front view of a cartridge receptacle disengaged from a cap (A), and a top view of a cartridge receptacle disengaged from a cap (B).

FIG. 4 shows an exploded perspective view of a cartridge of this disclosure.

FIG. 5 shows a perspective view of one embodiment of a system of this disclosure.

FIG. 6 shows an enlarged view of the spindles and receiving area depicted in FIG. 5.

FIG. 7 shows a perspective view of a different embodiment of a device of this disclosure depicting, among other things, sheathes surrounding spindles (A), and a close up of the sheathes and spindles (B).

FIG. 8 shows bar graphs quantifying the viability and yield of cell suspensions obtained after dissociating spleen ((A) and (B)), brain ((C) and (D)) and lung ((E) and (F)) tissues using an automated system of the present disclosure compared to two commercially available (automated and manual) tissue dissociation systems. Data represent the mean of 2-13 experiments. Also shown are bar graphs quantifying the effect of temperature differences during a lung dissociated protocol on viability (G) and yield (H) of arising cells.

### DETAILED DESCRIPTION

This disclosure relates to systems, apparatus and methods for processing a biological sample. In one embodiment the systems, apparatus and methods of this disclosure are automated. In one embodiment, the systems, apparatus and methods are used to process a biological sample to yield a suspension of cells, such as a single cell suspension. In one embodiment, the systems, apparatus and methods are used to process a biological sample to yield a tissue homogenate. In one embodiment, the systems, apparatus and methods are used to process a biological sample to yield a suspension of cells, such as a single cell suspension and/or a tissue homogenate.

Processing of a biological specimen or sample generally results in a reduction in its complexity. In one embodiment, processing a biological specimen or sample results in its dissociation. Where used herein, the term “dissociation” refers to reducing the complexity or organization of a biological sample, such as a tissue or tumour, into a plurality of cells. Preferably, a majority of the dissociated cells are intact and/or viable. Generally, when dissociating a biological sample, such as a tissue or tumour, into a suspension of cells it may be desirable to limit or avoid the homogenization of the biological sample, including the cells.

In one embodiment, processing a biological specimen or sample results in its homogenization. Where used herein, the term “homogenization” refers to a more significant (relative to dissociation) reduction in the complexity or organization of a biological sample, such as a tissue or tumour, into a suspension comprising a plurality of subcellular components, such as organelles, nucleic acids, and other subcellular components. Generally, when homogenizing a biological sample, such as a tissue or tumour, into a suspension of subcellular components it may be desirable to completely (or substantially completely) break apart cells but to limit or avoid the breakdown of organelles and other macromolecules.

Biological samples to be processed using the systems, apparatus and methods of this disclosure are not particularly limited as long as they comprise cells. In one embodiment, a biological sample corresponds to any tissue or any tissue fragment. By way of non-limiting example, the tissue may be a brain, liver, pancreas, spleen, prostate, lung, or a portion thereof. In one embodiment, a biological specimen corresponds to a tumour or a tumour sample.

After dissociating a biological sample using the disclosed systems, apparatus and methods, it may be desirable to perform downstream experiments, cultures or assays using the dissociated cells. In such cases, it is preferred that a substantial fraction of the recovered cells are viable. Thus, it may be important to process a freshly isolated rather than a preserved (such as cryopreserved) biological sample using the disclosed systems, apparatus and methods. However, not all applications are done using live cells, such as when cell staining is to be performed or when the recovery of subcellular components or macromolecules is desired.

After homogenizing a biological sample using the disclosed systems, apparatus and methods, it may be desirable to perform downstream experiments, cultures or assays using the subcellular components, such as nucleic acids or proteins. In such cases, it is preferred that a substantial fraction of the cells of the tissue have been broken apart. It may, nevertheless, be important to homogenize a freshly isolated rather than a preserved (such as cryopreserved) biological sample using the disclosed systems, apparatus and methods.

In one embodiment, the disclosed systems, apparatus and methods are preferably used to process a biological sample, such as a tissue, tumour, or portion thereof, to obtain a suspension of cells. Accordingly, it is advantageous to use systems, apparatus and methods that achieve a high yield of cells, maximize the frequency of viable cells, and/or minimize the homogenization of cells.

### Cartridge

In one aspect of this disclosure are provided cartridges for processing a biological sample. A cartridge of this disclosure may be used on its own or in conjunction with a system of this disclosure. In one embodiment, a cartridge of this disclosure interfaces with an instrument or a system of this disclosure. A cartridge (or a cartridge interfaced with a system) may be used to carry out a processing protocol on a biological sample, such as a mincing, dissociation or homogenization protocol.

A cartridge of this disclosure may be single-use or may be re-usable. In some embodiments, it is preferable that a cartridge is single-use, for example when the cartridge is used to process a biohazard or when it is used in regulated workflows. In some embodiments, it is preferable that a cartridge is re-usable, and in such embodiments the cartridge may be washable and/or sterilizable. In one embodiment, a cartridge of this disclosure may at a user's option be used only once or re-used.

A cartridge of this disclosure is not particularly limited in terms of the material(s) from which it is made. A cartridge may be sufficiently rigid in order to withstand reasonable wear and tear. In one embodiment, a cartridge is made of a polymer and may be manufactured by injection molding or 3D printing. In such an embodiment, a cartridge may be transparent or translucent, to allow a user to observe the progress of biological sample processing without the need to open the cartridge. In one embodiment, a cartridge is die cast, such as out of stainless steel.

The material from which a cartridge is made should be capable of withstanding a range of temperatures, such as from −80° C. to 80° C. However, the material from which a cartridge is made should not leach and should not break down when exposed to the biological sample or a solution in which the biological sample is bathed, whether at ambient temperature or a different temperature. Further, the material from which a cartridge is made should not be toxic to a biological sample being processed.

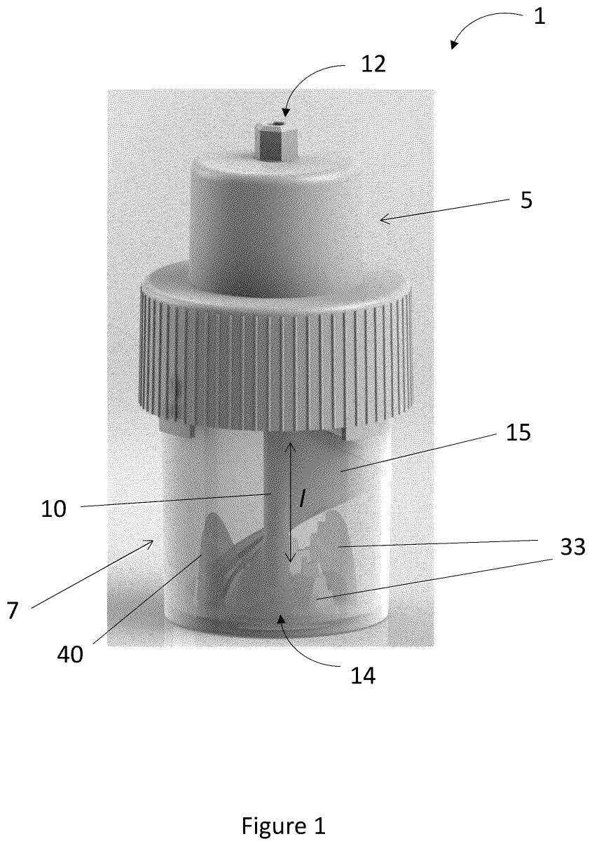

In one embodiment, a cartridge 1 of this disclosure may comprise multiple parts, such as a cap 5 and a receptacle 7. With reference to FIGS. 1-4, a cartridge for processing a biological sample 1 comprises a cap 5 having an upper side (exposed to an external environment) and an underside (substantially protected from an external environment when engaged with a receptacle).

Cap 5 may be secured to a complementary receptacle 7 in many different ways. In one embodiment, cap 5 is threaded for engaging receptacle 7, preferably near an opening thereof. In one embodiment, a ridge on either of the cap or receptacle is mateable with a groove on the cap or receptacle, such as by press fitting.

Cartridge 1 further comprises a shaft 10 having a first end 12 and an opposed second end 14, defining an axis I. First end 12 cooperates with cap 5 and second end 14 extends away from an underside of the cap. In one embodiment, first end 12 is received through a bore 8 in cap 5. In one embodiment, first end 12 cooperates with the underside of cap 5.

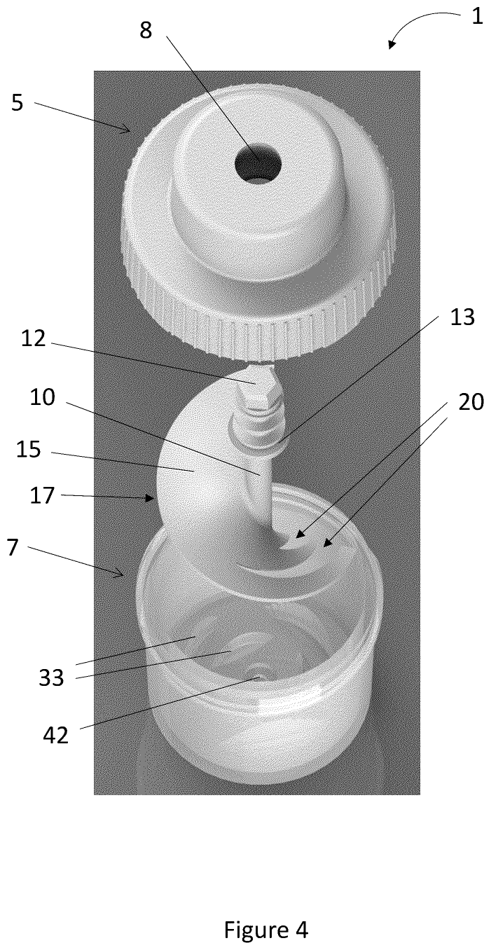

Shaft 10 and cap 5 are rotatable relative to one another. In one embodiment, an axis of rotation is defined by longitudinal axis I of shaft 10 (e.g. the axis defined through the first end 12 and opposed second end 14). Although rotatable relative to one another, it may be important that shaft 10 and cap 5 form a leak proof seal. In one embodiment, shaft 10 (and/or first end 12) and cap 5 are in a sealed (or leak-proof) engagement that nevertheless permits rotation of shaft 10 within bore 8. Means of sealing relatively movable components are known, and may include a grommet, a gasket, a bearing, or the like (shown as seal 13 in FIG. 4).

Cartridge 1 further comprises agitator 15 attached to shaft 10. Agitator 15 may be a separate part connected to shaft 10 or may be integral with shaft 10. Agitator 15 rotates within cartridge 1. In one embodiment, agitator 15 rotates independently about axis I (as defined by shaft 10). In one embodiment, agitator 15 rotates together with shaft 10 about axis I (as defined by shaft 10).

Agitator 15 may take any form provided that it is capable of directing movement of a fluid or a substance (such as a biological sample or a fragment of a biological sample) that comes into contact therewith. In one embodiment, agitator 15 may both move a substance that comes into contact therewith and direct the substance in a specific direction (e.g. downward toward a bottom wall of the receptacle or across a cutting surface). In one embodiment, agitator 15 is an impeller. In one embodiment, agitator 15 is a fin. In such embodiments, agitator 15 may flare outward of shaft 10. In one embodiment, agitator 15 flares laterally from shaft 10.

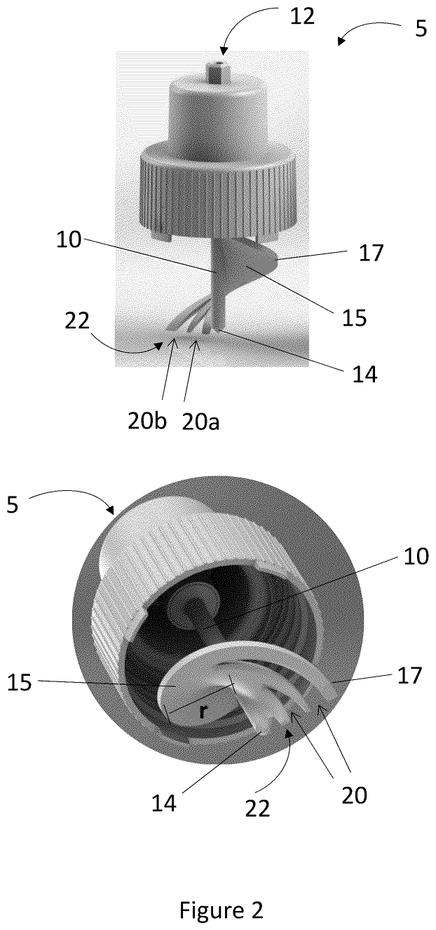

In one embodiment, agitator 15 comprises a lateral or outward edge 17 (see FIGS. 2 and 4). Lateral edge 17 refers to the portion of agitator 15 that is furthest away from shaft 10 as measured in the plane orthogonal to shaft 10, and more specifically to axis I.

In one embodiment, agitator 15, and more specifically lateral edge 17, flares radially wider in a direction from first end 12 to second end 14.

In one embodiment, a distance r of lateral edge 17 from shaft 10 or axis I (taken in the plane orthogonal to axis I) is constant or substantially constant along a length of agitator 15 (e.g. along axis I defined by first end 12 and second end 14). A constant or substantially constant extent of lateral edge 17 may be important to minimize gaps between lateral edge 17 (of agitator 15) and an interior (side) wall of receptacle 7, so as to reduce or limit the escape of biological sample from contact with agitator 15.

Agitator 15 may further comprise one or more slots 20 (which may be referred consecutively as 20a, 20b, and so on) in a peripheral edge 22 thereof (see FIG. 2). In one embodiment, peripheral edge 22 extends along a plane orthogonal to axis I. In one embodiment, the plane along which peripheral edge 22 extends (along distance r) is in the same plane as a plane orthogonal to a tip of second end 14. In one embodiment, the plane along which peripheral edge 22 extends is parallel to a plane orthogonal to a tip of second end 14. In one embodiment, the plane along which peripheral edge 22 extends is lower than a plane orthogonal to a tip of second end 14, that is to say that peripheral edge 22 extends further away from first end 12 than does a tip of second end 14. In one embodiment, the plane along which peripheral edge 22 extends is higher than a plane orthogonal to a tip of second end 14, that is to say that a tip of second end 14 extends further away from first end 12 than does peripheral edge 22. In one embodiment, peripheral edge 22 is the same edge as lateral edge 17.

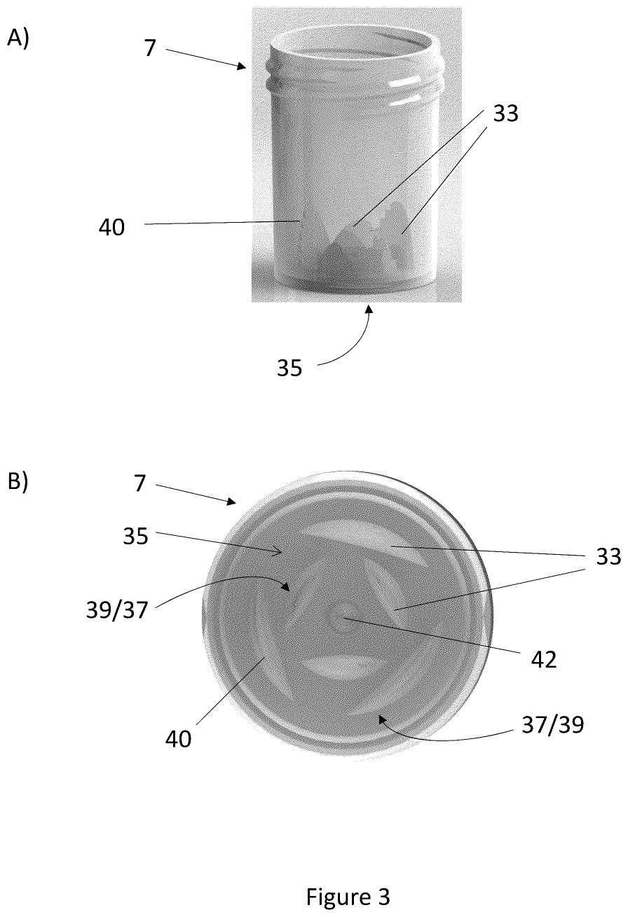

With reference to FIG. 3, a cartridge for processing a biological sample 1 further comprises a receptacle 7 engageable with an underside of cap 5. Receptacle 7 may be made of any material, provided that it is capable of containing a liquid and a solid. In one embodiment, receptacle 7 may be made of the same material as cap 5. In one embodiment, receptacle 7 may be made of a material that is different from a material used to make cap 5. Regardless of whether cap 5 and receptacle 7 are made of the same or different material, it is important that the two components are mateable, connectable, or attachable.

In one embodiment, cap 5 and receptacle 7 are sealed when engaged or mated with one another.

Receptacle 7 may comprise one or more teeth 33 (may be referred consecutively as 33a, 33b, and so on), arranged in an interior wall 35 thereof. In one embodiment, receptacle 7 comprises a plurality of teeth arranged in an interior wall 35 thereof. In one embodiment, receptacle 7 comprises a single tooth arranged in an interior wall 35 thereof.

In one embodiment, interior wall 35 is a bottom wall of receptacle 7. In one embodiment, interior wall 35 is a side wall of receptacle 7. The wall of receptacle 7 in which one or more teeth 33 are arranged will depend on where one or more slots 20 are configured on agitator 15. If one or more slots 20 are configured in lateral edge 17, then one or more teeth 33 are arranged in a sidewall of receptacle 7, particularly in an inner sidewall of receptacle 7. If one or more slots 20 are configured in peripheral edge 22, then one or more teeth 33 are arranged in a bottom wall of receptacle 7, particularly in an inner bottom wall of receptacle 7.

One or more teeth 33 may project toward an interior of receptacle 7. In one embodiment, one or more teeth 33 project orthogonal to interior wall 35 and toward an interior of receptacle 7. In one embodiment, one or more teeth 33 project at an angle that is not orthogonal to interior wall 35 and toward an interior of receptacle 7.

The angle, whether orthogonal or otherwise, at which one or more teeth 33 project toward an interior of receptacle 7 is not particularly limited, as indicated above. However, it is important that whatever angle one or more teeth 33 project away from interior wall 35, they pass through one or more slots 20, such as when shaft 10 (and/or agitator 15) rotate about axis I.

In one embodiment, plurality of teeth 33 are annularly arranged in interior wall 35 of receptacle 7, such as in a bottom wall thereof. In one embodiment, plurality of teeth 33 are annularly or concentrically arranged in at least a first bank of teeth 37 and a second bank of teeth 39. In one embodiment, first bank of teeth 37 are positioned radially outward of second bank of teeth 39. In one embodiment, first bank of teeth 37 are positioned radially inward of second bank of teeth 39. If receptacle 7 comprises more than two banks of annularly or concentrically arranged teeth, then such additional banks of teeth may be positioned in sequence with respect to first bank of teeth 37 and second bank of teeth 39.

In one embodiment, plurality of teeth 33 are circumferentially arranged in interior wall 35 of receptacle 7, such as in a sidewall thereof (not shown). In one embodiment, plurality of teeth 33 are circumferentially arranged in at least a first bank (e.g. row) of teeth 37 and a second bank (e.g. row) of teeth 39. In one embodiment, first bank (e.g. row) of teeth 37 are positioned closer to an opening of receptacle 7 than second bank (e.g. row) of teeth 39. In one embodiment, first bank (e.g. row) of teeth 37 are positioned farther from an opening of receptacle 7 than second bank (e.g. row) of teeth 39. If receptacle 7 comprises more than two banks (e.g. rows) of circumferentially arranged teeth, then such additional banks (e.g. rows) of teeth may be positioned in sequence with respect to first bank (e.g. row) of teeth 37 and second bank (e.g. row) of teeth 39.

In one embodiment, first bank of teeth 37 and second bank of teeth 39 project to the same or substantially the same extent toward the interior of receptacle 7. In one embodiment, first bank of teeth 37 extend further toward the interior of receptacle 7 than second bank of teeth 39. In one embodiment, second bank of teeth 39 extend further toward the interior of receptacle 7 than first bank of teeth 37.

The shape of the one or more teeth 33 is not particularly limited provided that they are dimensioned to (respectively) pass through one or more slots 20, such as when shaft 10 (and/or agitator 15) rotate about axis I. The following descriptions of exemplary tooth shapes are taken in a plane parallel to the interior wall in which they are arranged.

In one embodiment, a cross section of the one or more teeth 33 (or the plurality of teeth) is an ellipse. In such embodiments, an eccentricity of the ellipse is greater than 0 and less than 1. In one embodiment, an eccentricity of the ellipse is about 0.5 or greater (and less than 1).

In one embodiment, a cross section of the one or more teeth 33 (or the plurality of teeth) is curved or arcuate. In one embodiment, a curved or arcuate cross-sectional shape may correspond to the radius of curvature of annulus in which one or more teeth 33 may be arranged.

In one embodiment, a cross section of the one or more teeth 33 (or the plurality of teeth) is rounded or circular or ovular. In one embodiment, a cross section of the one or more teeth 33 (or the plurality of teeth) is a different polygon, such as a quadrilateral, a pentagon, hexagon, rhombus or the like. In such embodiment, as with all embodiments, the only constraint on the shape and height of the teeth is that they respectively pass through one or more slots 20, such as when shaft 10 (and/or agitator 15) rotate about axis I.

Processing of a biological sample, such as dissociation or homogenization, may be facilitated through design features included on plurality of teeth 33. In one embodiment, one or more teeth 33 terminate in or comprise a tapered edge 40. Tapered edge 40 may facilitate the breakdown of a biological sample contained in receptacle 7, particularly as agitator 15 moves the biological sample into proximity of plurality of teeth 33 as it rotates about axis I. In one embodiment, tapered edge 40 is smooth. In one embodiment, tapered edge 40 is serrated. In one embodiment, a subset of plurality of teeth 33 are smooth and the remaining teeth are serrated.

Receptacle 7 may further comprise an alignment feature 42 for engaging second end 14 (or a tip thereof) of shaft 10. With second end 14 of shaft 10 engaged with alignment feature 42, it may be possible to ensure and maintain the rotation of agitator 15 about a consistent (non-wandering) axis I of rotation, while limiting or avoiding the improper alignment of the one or more teeth 33 and one or more slots 20.

In one embodiment, alignment feature 42 is positioned or configured in internal wall 35 of receptacle 7. In one embodiment, alignment feature 42 is positioned or configured in a bottom wall of receptacle 7. In such an embodiment, alignment feature 42 may be a dimple or depression in bottom wall. In one embodiment, alignment feature 42 is positioned or configured in a sidewall of receptacle 7. In such an embodiment, alignment feature 42 may be a circumferential groove in sidewall.

Where cartridge 1 is used for dissociating a biological sample, such as a tissue or tumour, the cartridge may be considered a dissociation cartridge. Where cartridge 1 is used for homogenizing a biological sample, such as a tissue or tumour, the cartridge may be considered a homogenization cartridge.

In one aspect, cartridge 1 may be provided in component parts (e.g. cap 5 separate from receptacle 7). In one aspect, cartridge 1 may be provided together or in assembled form (e.g. cap 5 engaged with receptacle 7).

### System

In one aspect of this disclosure are provided systems for processing a biological specimen or sample. In one embodiment the systems are automated. In one embodiment, the systems interface with a cartridge as described above.

System 100 (in conjunction with cartridge 1) may be used to dissociate and/or homogenize a biological sample, such as a tissue or tumour sample.

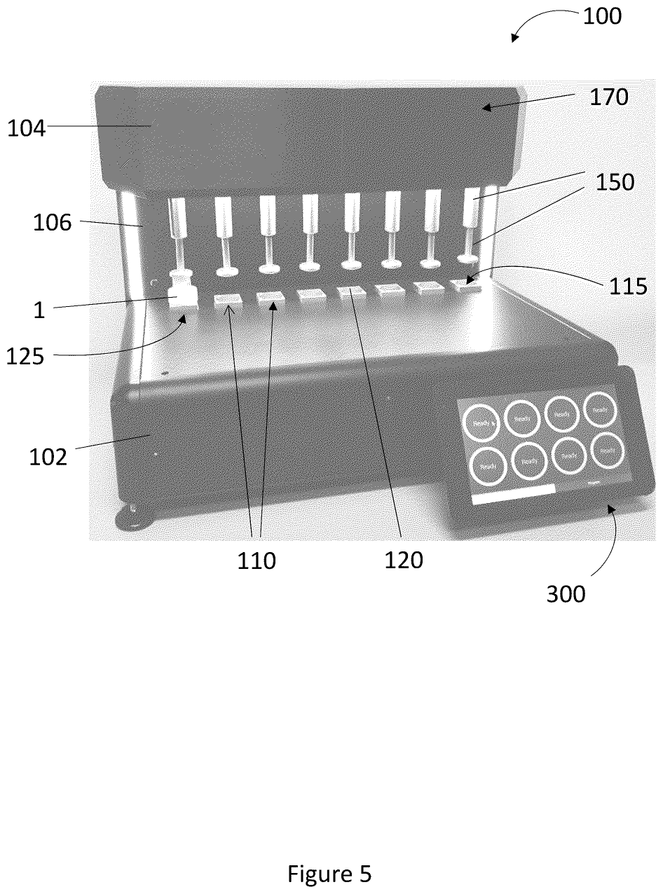

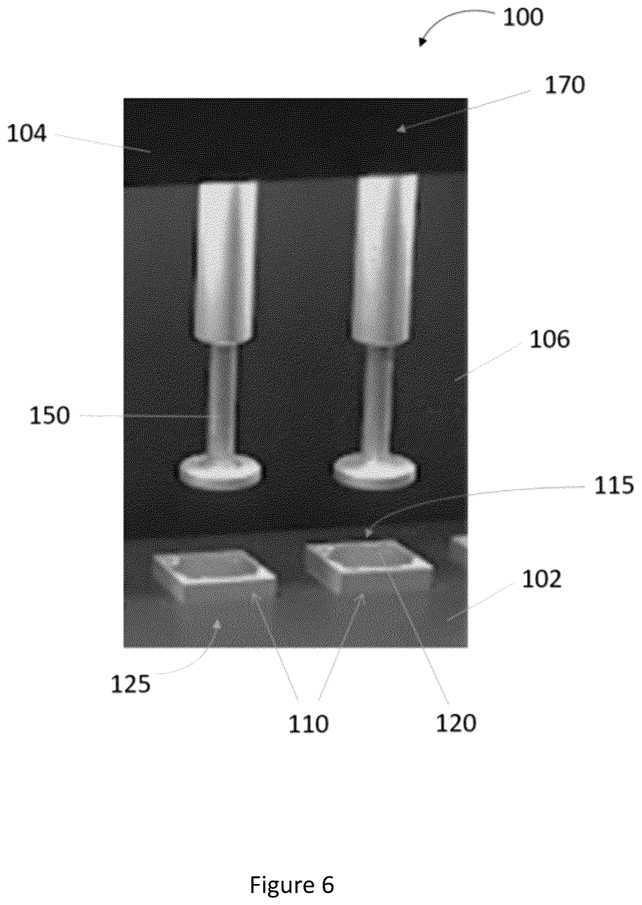

With reference to FIGS. 5 to 7, system 100 may comprise a base 102 and a head 104 connected to the base by a back wall 106.

Base 102 may comprise one or more receiving areas 110 for respectively receiving a cartridge 1 (e.g. an assembled cartridge 1, and more specifically receptacle 7 thereof). In one embodiment, base 102 comprises a plurality of receiving areas 110 for respectively receiving a cartridge 1 (e.g. an assembled cartridge 1, and more specifically receptacle 7 thereof). In one embodiment, base 102 comprises 2 receiving areas 110, 3 receiving areas 110, 4 receiving areas 110, 5 receiving areas 110, 6 receiving areas 110, 7 receiving areas 110, 8 receiving areas 110, 9 receiving areas 110, 10 receiving areas 110, 11 receiving areas 110, 12 receiving areas 110, or more.

Each receiving area 110 may comprise a bore 115 circumscribed by a bore wall 120. A cross-sectional shape of bore 115 is not particularly limited provided that it accommodates receptacle 7 and that rocking or lateral movement of receptacle 7 within bore 115 is avoided or limited (such as by a sufficiently deep bore 115 relative to a height of receptacle 7). In some embodiments, a cross-sectional shape of bore 115 is the same or substantially the same as a cross-sectional shape of receptacle 7 (both taken in a plane defined by 0 and 180 degrees). In some embodiments, a cross-sectional shape of bore 115 is different from or larger than a cross-sectional shape of receptacle 7. In such embodiments, it may be important to use an adapter or to configure guide features within bore 115 to control rocking or lateral movement of receptacle 7 therein.

Bore 115 may be dimensioned with a diameter or width about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.5 mm, about 2 mm, about 2.5 mm wider/larger than a diameter of receptacle 7 of cartridge 1. In one embodiment, bore 115 may be dimensioned with a diameter or width about 2.5 mm wider/larger than a diameter of receptacle 7 of cartridge 1.

In one embodiment of system 100, one or more receiving areas 110 are present in a fixed position within base 102. In one embodiment of system 100, one or more receiving areas 110 are movable (e.g. rotatable) within base 102, such as on a carousel integrated into or on base 102.

Base 102 may further comprise one or more Peltier module 125. In one embodiment, each bore 115 is respectively associated with a Peltier module 125. Thus, in one embodiment, system 100 comprises the same number of Peltier modules as receiving areas 110 (and bores 115). While the following description may apply to each of one or more Peltier module 125, for simplicity only a single Peltier module 125 will be referenced.

In one embodiment, Peltier module 125 is incorporated into base 102 and establishes or adjusts a temperature of at least a portion of base 102, such as bore wall 120. In one embodiment, Peltier module 125 is communicatively coupled to at least one processor or microprocessor. Thus a temperature of base 102, and more specifically of receiving area 110, and still more specifically of bore wall 120, is controllable under a user selected or input sample processing protocol.

System 100 further comprises one or more spindles 150 connected to head 104. One or more spindles may engage or be engageable with shaft 10 of cartridge 1 (when cartridge 1 is positioned in receiving area 110 and/or bore 115), and more particularly with first end 12 thereof. In one embodiment, one or more spindles 150 depend from head 104. In one embodiment, the number of spindles correspond to the number of receiving areas 110, and more specifically to the number of bores 115.

In one embodiment, the one or more spindles 150 are rotatable. While the following description may apply to each of one or more spindles 150, for simplicity only a single spindle 150 will be referenced. In one embodiment, each spindle 150 rotates about the same axis I as shaft 10 of cartridge 1.

In embodiments of system 100 comprising rotatable spindles, system 100 comprises one or more motors 170, such as a stepper motor. Typically, one or more motors 170 are positioned within head 104. In one embodiment, a respective motor imparts rotational motion to a respective spindle 150. In one embodiment, a single motor imparts rotational motion to a plurality of spindles 150.

In one embodiment, spindle 150 itself is not rotatable but rather comprises a transmission that imparts rotational motion to shaft 10 (or agitator 15). In one embodiment, spindle 150 itself is not rotatable but rather comprises wiring in communication with a rotor assembly that imparts rotational motion to shaft 10 (or agitator 15).

Spindle 150 may be movable or biasable between a first retracted position and a second extended position. Thus, spindle 150 may cooperate with receiving area 110 (and bore 115) to accommodate cartridges 1 of different heights and/or different shaft 10 lengths.

Engagement or mating of spindle 150 with shaft 10 (and more particularly with end 12), whether direct or indirect, may cause agitator 15 to rotate under the influence of a motor, such as motor 170. The means by which spindle 150 and shaft 10 become engaged or mated is not particularly limited. In some embodiments, engagement or mating of spindle 150 and shaft 10 is by complementary male and female connection. The male connector may be localized to spindle 150 and the female connector may be localized to shaft 10 (or more specifically to end 112), or vice versa.

Regardless, the male and female connecting counterparts are shaped in a way that is complementary and also in a way that avoids or limits slippage therebetween, such as when spindle 150 is rotated. In one embodiment, when viewed in the plane orthogonal to longitudinal axis I of shaft, the male connector comprises at least one straight edge that is mateable with a correspondingly shaped female connector counterpart. In one embodiment, the male connector comprises more than one straight edge that is mateable with a correspondingly shaped female connector counterpart. By way of non-limiting example, the male connector may be semicircular, triangular, square, pentagonal, hexagonal, or any other shape comprising at least one straight edge. While a round or rounded shape may slip or eventually slip when torque is applied thereto, a connection mediated by at least one straight edge or more than one straight edges may better tolerate higher levels of torque without resulting in relative slipping of the two connectors.

In a specific embodiment, a male connector comprising at least one, or more than one straight edge (when viewed in a plane orthogonal to a longitudinal axis of shaft 10) projects from shaft 10 (or end 12) in the direction of spindle 150, and spindle 150 comprises a female connector that is mateable with (e.g. complementary to) the male connector (see FIGS. 4 and 5).

In a specific embodiment, a female connector comprising at least one, or more than one straight edge (when viewed in a plane orthogonal to a longitudinal axis of shaft 10) is configured in shaft 10 (or in end 12) and spindle 150 comprises a male connector projecting in the direction of shaft 10 (or end 112) that is mateable with (e.g. complementary to) the female connector.

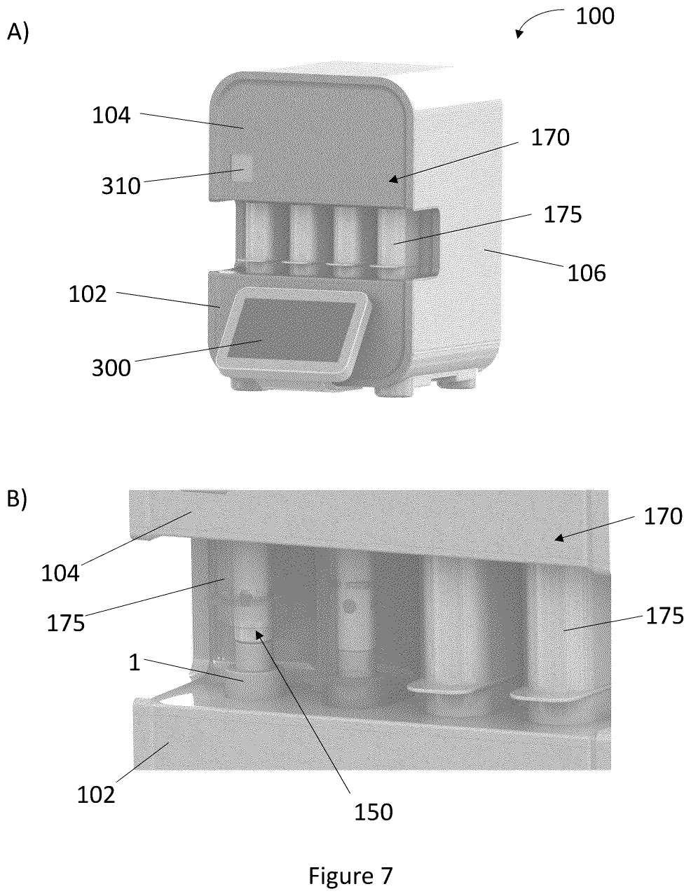

System 100 may further comprise a sheath 175 that surrounds or at least partially surrounds spindle 150 (FIG. 7). Similar to spindle 150, sheath 175 may be movable or biasable in between a first retracted position and a second extended position. Sheath 175 may retract completely into head 104, or only partially, and sheath 175 may extend toward or to base 102. In one embodiment, spindle 150 and an associated sheath 175 are capable of movement (along axis I) relative to one another.

In one embodiment, spindle 150 and an associated sheath 175 are capable of movement (along axis I) relative to one another but only along a limited path until an engagement feature of sheath 175 contacts an upper limit or lower limit of spindle 150 (not shown). By way of further description, as sheath 175 is moved upward into a retracted position, an engagement feature on an inwardly facing surface of sheath 175 contacts an upper limit on spindle 150 causing the spindle to simultaneously retract together with sheath 175. And, as sheath 175 is moved downward into an extended position, an engagement feature on an inner surface of sheath 175 contacts a lower limit on spindle 150 causing the spindle to simultaneously extend together with sheath 175. Thus, a single movement of sheath 175 upward or downward along axis I may extend or retract both sheath 175 and an associated spindle 150.

In embodiments where system 100 is automated, high amounts of heat may be generated during operation, particularly when both of one or more Peltier module 125 and one or more motor 170 are running. To alleviate the buildup of heat to high or unsafe levels, venting may be provided in system 100, such as in base 102 and/or in head 104 and/or in back wall 106 and/or in side panels. Venting may comprise a single vent or a plurality of vents/perforations. In one embodiment, venting/perforations may be provided in a bottom wall of base 102. In one embodiment, venting/perforations may be provided in one or more side walls of base 102. In one embodiment, venting/perforations may be provided in both a bottom wall and one or more side wall of base 102. In one embodiment, venting/perforations may be provided in back wall 106 (or panel) of system 100. In one embodiment, venting/perforations may be provided in head 104 substantially overlapping where one or more motors 170 are positioned.

In one embodiment, venting may be provided as a slit in head 104 substantially overlapping where one or more motors 170 are positioned. In the same or different embodiment, venting or a component of venting may be provided as a slit or perforations in base 102 substantially overlapping where one or more Peltier module(s) 125 are positioned.

System 100 may further comprise internal ducting (not shown). In one embodiment, ducting is in fluid communication with an internal cavity of base 102.

Venting and/or ducting may cooperate with other cooling features of system 100, such as one or more fans, to bring external air inside of system 100 and move heated air out of system 100. In one embodiment, system 100 may comprise a fan configured to draw external air into system 100 and to move it through and out of system 100 via venting. In one embodiment, system 100 comprises a series of fans within base 102 and/or head 104 and/or back wall 106.

Thus, the cooperation of one or more fans and venting (e.g. slits and/or perforations) reduces the build-up of heat to ensure the safe operation of the various thermoelectric modules of system 100 and that they do not overheat.

In embodiments where system 100 is automated, it comprises at least one processor or microprocessor. The at least one processor may be configured to output a processing protocol to at least one controller or microcontroller. In one embodiment, one or more processing protocols may be pre-programmed into system 100. In one embodiment, one or more processing protocols may be input into system 100 by a user. In one embodiment, system 100 may comprise both of one or more pre-programmed processing protocols and customizable and/or inputtable processing protocols.

A user may select or input one or more processing protocols via a graphical user interface 300. In one embodiment, the one or more processing protocols may comprise in any sequence: one or more incubation steps; one or more temperature adjustments; and one or more agitation steps. Thus, a processing protocol may define the steps to be taken to dissociate or homogenize a biological sample.

Graphical user interface 300 may be comprised in system 100 or may be external of system 100. In one embodiment, graphical user interface 300 is connected to an internet or mobile communication network. In such embodiment, a user may select or input a processing protocol remotely, such as via a mobile device application. Graphical user interface may be a computer or tablet, and thus comprise at least one processor or microprocessor that is communicatively coupled to at least one controller or microcontroller (that directs operation of at least one or more motor 170 and one or more Peltier module 125).

A processing protocol may comprise setting or adjusting one or more of: a speed of rotation of motor 170 (and correspondingly of agitator 15); a duration of rotation of motor 170 (and correspondingly of agitator 15); and a direction of rotation of motor 170 (and correspondingly of agitator 15). In one embodiment, a processing protocol comprises, depending on the stage of a protocol, setting or adjusting two or each of a speed of rotation of motor 170 (and correspondingly of agitator 15), a duration of rotation of motor 170 (and correspondingly of agitator 15), and a direction of rotation of motor 170 (and correspondingly of agitator 15).

In some embodiments, a processing protocol may comprise establishing or modifying a temperature of bore wall 120. Establishing or modifying a temperature of bore wall 120 may be important when an enzyme solution is included in receptacle to facilitate processing of the biological sample therein, and the enzyme solution optimally performs at a specific temperature or temperature range. Thus, it may be important in such cases that receptacle 7 is in close association with bore wall 120, and that receptacle 7 is made of a material that readily conducts or radiates externally applied temperature. Establishing or modifying a temperature of bore wall 120 may also be important when a processed biological sample requires incubation at a specific temperature, such as at about 4° C. to preserve a processed sample before a user may collect it, and/or at about 37° C. for optimal enzyme activity.

In operation, a dissociation or homogenization or incubation protocol comprises various steps in sequence that may include one or more incubations (at one or more pre-determined temperatures) and one or more agitation steps (at one or more pre-determined agitation rates and/or directions and/or durations).

System 100 may further comprise at least one sensor downstream of the at least one processor or microprocessor to relay a feedback signal to the at least one processor or microprocessor, so that a corrective action may be undertaken. Exemplary signals include but are not limited to a temperature inside system 100, a temperature of bore wall 120, a mass load on or in a receiving area 110 and/or bore 115, or a proper connection between spindle 150 and shaft 10.

Regardless of the feedback signal, the at least one processor or microprocessor may be programmed to undertake a corrective action or to abort a processing protocol. For example, if a temperature of system 100 and/or bore wall 120 is outside of an acceptable range, then the processing protocol will not execute until a user has intervened or the temperature falls to within an acceptable range. As an another example, if a mass load on or in a receiving area 110 and/or bore 115 is outside of an acceptable range, then the processing protocol will not execute until a user has intervened or the mass load falls within an acceptable range. As another example, if sheath 175 is not fully extended, then the processing protocol will not execute until a user has intervened or the mass load falls within an acceptable range. As another example, if spindle 150 is not properly engaged with shaft 10, then the processing protocol will not execute until a user has intervened or the mass load falls within an acceptable range.

System 100 may be modular in so far as multiple systems may be connected or daisy chained to one another, so as to increase or reduce the number of receiving areas for cartridges 1. Likewise system 100 may be module in so far as a number of receiving areas of a single system may be increased or reduced.

In one embodiment, a primary system (as described above) comprises at least one processor (or microprocessor) and at least one controller (or microcontroller), and a secondary system daisy chained thereto also comprises at least one processor (or microprocessor) and at least one controller (or microcontroller).

In one embodiment, a primary system (as described above) comprises at least one processor (or microprocessor) and at least one controller (or microcontroller), and a secondary system daisy chained thereto does not comprise at least one processor (or microprocessor). Rather, secondary system may rely on the at least one processor (or microprocessor) of a primary system to communicate a processing protocol thereto (and more particularly to at least one controller (or microcontroller) thereof). Thus, processing protocols for each of the primary system and the secondary system may be selected or input via a single interface, such as interface 300.

System 100 (whether primary, secondary and so on) may comprise a secondary display 310, to feedback or cross-reference certain information to a user, such as a system or spindle/receiving area identifier.

In one embodiment, regardless of whether only a primary system has been deployed or a primary and secondary system are connected to one another, a user may execute different processing protocols on each system. In one embodiment, regardless of whether only a primary system has been deployed or a primary and secondary system are connected to one another, a user may execute different processing protocols on each cartridge placed in a receiving area of a respective system.

### Methods

In one aspect of this disclosure are provided methods for processing a biological sample. Biological samples may be processed, dissociated, and/or homogenized using cartridges (as described above) and/or systems (also as described above). In one embodiment, a biological sample is processed, dissociated, and/or homogenized using a cartridge (as described above) that interfaces with a system (as described above).

Methods of this disclosure may be carried out on any biological sample comprising cells. In one embodiment, the biological sample is an organ, or a portion of an organ. In one embodiment, the biological sample is a tumour, or a portion thereof. However, the types of biological samples that may be processed, dissociated, and/or homogenized using a cartridge and/or a system of this disclosure are not necessarily limited to samples obtained from a subject or patient. For example, the biological sample may be a soil sample comprising unicellular and multicellular organisms, or a sample of plant matter.

Providing a biological sample in a cartridge (as described above) may encompass suspending the biological sample in an appropriate solution. Solutions used to suspend a biological sample are not particularly limited, but in some embodiments it is preferable that such a solution is non-toxic to the biological sample (and an arising cell suspension or homogenate). In one embodiment, a solution used to suspend a biological sample is essentially isosmotic with the cells, organelles, or other membrane-found components comprised in the biological sample.

A biological sample may be suspended or incubated in a solution comprising one or more enzymes. Enzymes for breaking down tissues and tumours (e.g. dissociating and/or homogenizing) are widely known and commercially available. One or more enzymes comprised in a solution may be selected from the group consisting of serine proteases, cysteine proteases, aspartic proteases and metalloproteases. In addition or in the alternative, one or more enzymes comprised in a solution may include one or more of trypsin, chymotrypsin, elastase, dispase, thermolysin, collagenase, hyaluronidase, papain, calpain, lysosomal cathepsin, pepsin, subtilisin, rennin and carboxypeptidases.

In one embodiment, the enzymes may comprise enzymes that possess a catalytic activity at temperatures above 25° C. In one embodiment, the enzymes may comprise enzymes that possess a catalytic activity at temperatures below 25° C. In one embodiment, the enzymes may comprise cold-active enzymes produced by cold-adapted microbes. In one embodiment, the cold-active enzymes may comprise proteases.

If yielding a processed biological sample comprises yielding a dissociated suspension of (intact and/or viable) cells, careful selection of enzymes and their concentrations may be important, so as to avoid digesting or cleaving, for example, proteins or glycoproteins associated with cell membranes.

As described above, rotating or oscillating agitator may bring a biological sample into contact with one or more teeth of a consumable, thus causing the biological sample to become reduced in complexity. Depending on the rate, duration and direction of rotation or oscillation of shaft (and agitator), the reduction in complexity of the biological sample may range from minced, to dissociated, to homogenized.

In one embodiment, rotating or oscillating the shaft (and agitator) is effected in a system (as described above), by (biasably) engaging/mating one or more spindles with a respective shaft end of a cartridge. In one embodiment, rotating or oscillating the agitator is under the control of a (micro)processor, a (micro)controller and a motor.

In one embodiment, dissociating and/or processing and/or homogenizing a biological sample using a cartridge and system of this disclosure may require a user to select or input a protocol, such as via a graphical user interface integrated with the system or via a mobile application.

In one embodiment, after selecting an appropriate protocol the methods may yield a processed biological sample wherein the dissociated cells exhibit a high degree of viability, such as >70%, >75%, >80%, >85%, >90%, or >95% viable cells.

In one embodiment, after selecting an appropriate protocol the methods may yield a processed biological sample wherein a high degree of cells are recovered, such as per milligram of tissue processed. In one embodiment, >1000 cells are recovered per milligram of tissue processed. In one embodiment, >5000 cells are recovered per milligram of tissue processed. In one embodiment, >10000 cells are recovered per milligram of tissue processed. In one embodiment, >15000 cells are recovered per milligram of tissue processed. In one embodiment, >20000 cells are recovered per milligram of tissue processed. In one embodiment, >25000 cells are recovered per milligram of tissue processed. In one embodiment, >30000 cells are recovered per milligram of tissue processed.

In one embodiment, after selecting an appropriate protocol (e.g. a dissociation protocol) the methods may yield a processed biological sample wherein a low degree of homogenization has occurred. In other words, a low number of cells are broken down and a high number of intact cells are present in the dissociated sample. A degree of homogenization may be determined by analyzing a quantity of one or more macromolecules in the processed sample, such as intracellular proteins, cell membrane fragments, or genomic DNA.

In one embodiment, the methods carried out using a cartridge and/or a system of this disclosure may result in comparable or improved yields of viable cells and/or recovered cells (such as per milligram of tissue) in comparison to other ways of processing a biological sample (e.g. manual processing by mortar and pestle, or using a different automated means).

An automated system that interfaces with a cartridge (both as described herein, and depicted in FIGS. 1 to 7) was evaluated for its biological sample processing performance. Spleen, lung, and brain samples harvested from C57BL/6 mice were respectively placed into a receptacle (as described above) in an appropriate solution and capped. An assembled cartridge was placed into a receiving area of an automated system of the present disclosure, and a processing protocol was selected (e.g. a dissociation protocol).

Additional, post-processing may be important in certain applications or for certain tissue or tumour types, such as to further clarify the processed sample. In one embodiment, additional DNase treatment may be important to reduce viscosity of a processed sample. In one embodiment, a processed sample may be passed through a filter to remove relatively (to the filter pore size) large particles and debris.

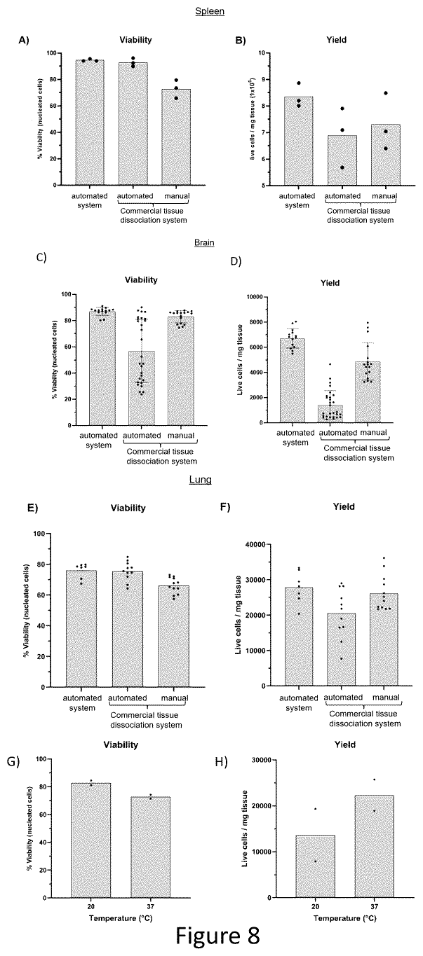

Dissociated cell suspensions may be subjected to further downstream assays or analyses. For example, a dissociated cell suspension may be subjected to cell separation or enrichment assays, using conventional approaches such as density gradient centrifugation, RBC lysis, or immunomagnetic separation. By way of another example, a dissociated cell suspension may be characterized by flow cytometry to assess viability and yield (FIG. 8). As can be seen in FIG. 8, methods of this disclosure may be comparable to or outperform other commercially available options for dissociating a sample.

For spleen samples processed by the described methods using a system and cartridge of this disclosure, more than 90% of dissociated cells were viable, and slightly lower viability was observed in a commercially available automated system. Indeed, markedly lower cell viability was observed among cells of a spleen sample dissociated manually (FIG. 8A). In terms of cell yield, notably higher levels were obtained using methods and systems of this disclosure in comparison to both commercially available approaches tested (FIG. 8B).

For brain samples processed by the described methods using a system and cartridge of this disclosure, more than 80% of dissociated cells were viable, and slightly lower viability was observed among cells dissociated using the two commercially available approaches tested (FIG. 8C). In terms of cell yield, notably higher levels were obtained using methods and systems of this disclosure in comparison to both commercially available approaches tested (FIG. 8D).

For lung samples processed by the described methods using a system and cartridge of this disclosure, nearly 80% of dissociated cells were viable, and the same or slightly lower viability was observed among cells dissociated using the two commercially available approaches tested (FIG. 8E). In terms of cell yield, higher levels were obtained using methods and systems of this disclosure in comparison to both commercially available approaches tested (FIG. 8F).

The effect of temperature differences during a lung dissociated protocol were also investigated. The results showed that while viability was slightly negatively impacted when performing a dissociation protocol at 37° C. relative to 20° C. (FIG. 8G), the yield of live cells was markedly higher when performed at 37° C. relative to 20° C. (FIG. 8H).

Therefore, the subject automated system demonstrated superior performance compared to commercially available tissue dissociation systems, with higher cell yields achieved for both brain and spleen tissues.

### Interpretation of Terms

While processes, steps or blocks are presented in a given order, alternative examples may perform routines having steps, or employ systems having blocks or steps, in a different order, and some processes or steps or blocks may be deleted, moved, added, subdivided, combined, and/or modified to provide alternative or sub-combinations. Each of these processes or steps or blocks may be implemented in a variety of different ways. Also, while processes or steps or blocks are at times shown as being performed in series, these processes or steps or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Where a component is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Various features are described herein as being present in “one embodiment” or in some “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. A cartridge for processing a biological sample, comprising:
a cap having an upper side and an underside;
a shaft rotatable relative to the cap, the shaft having a first end that cooperates with the cap and an opposed second end extending away from the underside;
an agitator attached to the shaft;
one or more slots in a peripheral edge of the agitator;
a receptacle engageable with the underside of the cap; and
one or more teeth arranged in an interior wall of the receptacle, the one or more teeth projecting toward an interior of the receptacle and passing through the one or more slots as the agitator rotates about an axis defined by the shaft.

2. The cartridge of claim 1, wherein the agitator is an impeller.

3. The cartridge of claim 1, wherein the agitator has a substantially constant radius.

4. The cartridge of claim 1, further comprising a plurality of teeth.

5. The cartridge of claim 4, wherein the plurality of teeth are radially arranged in at least a first bank of teeth.

6. The cartridge of claim 5, further comprising a second bank of teeth concentric with the first bank of teeth, wherein the first bank of teeth is positioned radially outward of the second bank of teeth.

7. The cartridge of claim 6, wherein the first bank of teeth project further toward the interior of the receptacle than the second bank of teeth.

8. The cartridge of claim 1, wherein the one or more teeth are arranged in a bottom wall of the receptacle.

9. The cartridge of claim 1, wherein a cross section of the one or more teeth taken in a plane parallel to the interior wall is an ellipse.

10. The cartridge of claim 9, wherein an eccentricity of the ellipse is greater than 0 and less than 1, and is preferably greater than 0.5.

11. The cartridge of claim 10, wherein the one or more teeth terminate in a tapered edge.

12. The cartridge of claim 8 or any other claim herein, further comprising an alignment feature in the bottom wall of the receptacle for engaging a tip of the second end of the shaft.

13. An assembled cartridge of claim 1, wherein the cap is engaged with the receptacle.

14. An automated system for processing a biological sample, comprising:
a base having one or more receiving areas for respectively receiving an assembled cartridge of claim 13;
at least one rotatable spindle engagable with the shaft of the assembled cartridge;
a motor for rotating the spindle; and
at least one processor or microprocessor configured to output a processing protocol to at least the motor.

15. The system of claim 14, wherein the at least one rotatable spindle
a) engages with the shaft of the assembled cartridge through a complementary and mateable connection; and/or
b) is movable or biasable between a first extended position to a second retracted position.

16. The system of claim 14, wherein the assembled cartridge is received within a receiving area comprising a bore circumscribed by a bore wall.

17. The system of claim 16, further comprising a Peltier module for establishing or modifying a temperature of the bore wall.

18. The system of claim 17, wherein the processing protocol comprises establishing or modifying a temperature of the bore wall.

19. The system of claim 14, wherein the processing protocol comprises setting the speed and/or direction and/or duration of rotation of the at least one spindle.

20. The system of claim 14, further comprising a sensor downstream of the at least one processor or microprocessor, the sensor relaying a feedback signal to the at least one processor or microprocessor.
